(19) United States
(12) Patent Application Publication
(10) Pub. No.: US 2007/0217945 A1
(43) Pub. Date: Sep. 20, 2007

(54) Fan-Driven Air Freshener

(76) Inventor: Raymond K. Selander, Hopewell Junction, NY (US)

Correspondence Address: INTERNATIONAL FLAVORS & FRAGRANCES INC., 521 WEST 57TH ST, NEW YORK, NY, 10019, US

(21) Appl. No.: 11/532,694

(22) Filed: Sep. 18, 2006

Related U.S. Application Data
(63) Continuation-in-part of application No. 10/682,051, filed on Oct. 9, 2003, now abandoned.

(51) Int. Cl. A61L 9/00; B32B 5/02

(52) U.S. Cl. 422/5; 422/110

(57) ABSTRACT

An air freshener that has a source of air freshening chemical with a fan that is controlled either by an optical device that senses light or a motion detector is disclosed. When a light is turned on or motion is detected, the fan will be activated for a predetermined time period. In certain embodiments, the fan will stop turning after a predetermined time. In certain preferred embodiments the source of air freshening chemical is disposed beneath the fan and allows fragrance to be delivered over time without the fan. The additional airflow provided by the fan causes more volatile fragrance chemicals to be removed from the source of air freshening chemical and admitted into the environment. Preferably, a microprocessor controls the fan so that a “burst mode” is created by controlling the frequency and intensity of the pulses of air freshener that are emitted. Also disclosed is a system for delivering a volatilized chemical integrated into a robotic vacuum cleaner. A reservoir of volatile chemicals, such as a fragrance, is volatized and exhausted by the fan stream through an exhaust duct. As the robotic vacuum cleaner carries out its automated routine, it adds a scent, antibacterial chemical or other chemical to the surface being cleaned, e.g., carpeting.

### STATUS OF RELATED APPLICATIONS

This application is a continuation-in-part of U.S. Ser. No. 10/682,051 filed on Oct. 9, 2003, now pending, which is a continuation-in-part of U.S. Ser. No. 11/069,864 filed on Mar. 1, 2005, now pending, the contents of which are hereby incorporated by reference.

### FIELD OF THE INVENTION

The present invention relates to fragrance delivery systems, and more particularly to active systems in which a fan suffuses the air in an environment with a chemical to mask or minimize objectionable odors.

### BACKGROUND OF THE INVENTION

Various devices are known that “freshen” air by adding a chemical to the air. In particular, off odors and malodors found in bathrooms are common. Various devices and chemicals that disinfect, i.e., kill odor-causing bacteria, or spray a perfume or fragrance to mask odors are know. Although many of these systems are passive and emit an air freshening compound into the air continuously, others use a fan to circulate the air freshening compound more rapidly and in higher concentration.

Currently available air fresheners with fans have various limitations. One limitation is that they do not deliver air freshening compounds effectively, primarily because the compound is delivered in intermittent bursts of varying intensity, or pulses, while the fan is operating. Additionally, currently available designs simply turn the fan on and off manually. If the fan is activated for a period of time beyond that needed the life of the fan and motor assembly is shortened unnecessarily, as is the battery life in battery-driven models. Moreover, air freshening chemicals volatilized by the fan are used up more quickly if the fan is either constantly running or running for a period of time longer than necessary.

U.S. Pat. No. 4,695,435—Spector discloses an air freshener device with a motor driven fan that is activated by a light being turned on, and is deactivated when the light is turned off.

U.S. Pat. No. 4,707,338—Spector discloses an air freshener device with a motor driven fan that is activated by a light being turned on, and is deactivated after a set period of time.

Neither of these prior art devices address the problems outlined above. Therefore, there remains a long-felt yet unmet need for providing enhanced levels of volatile air freshening or aroma chemicals in an effective and efficient manner. It would therefore be desirable to provide materials and methods that enhance the efficiency of fan driven air freshening systems. It would further be desirable to provide such improvements in a manner that permitted their application across a wide variety of situations and that permitted their implementation in a cost-effective manner.

Self-propelled, self-navigating sweeper-vacuum cleaners known as “robot” vacuum systems are currently in market. U.S. Pat. No. 6,809,490 discloses a control system for a mobile robot vacuum cleaner to effectively cover a given area by operating in a plurality of modes, including an obstacle following mode and a random bounce mode, as well as spot coverage, such as spiraling or other modes to increase effectiveness and ensure full coverage. U.S. Pat. No. 6,594,844 discloses a robot obstacle detection system including a robot housing which navigates with respect to a surface and a sensor subsystem having a defined relationship with respect to the housing and aimed at the surface for detecting the surface. Both of these patents herein incorporated by reference in their entirety as if fully set forth herein, and both are assigned to iRobot Corporation which markets a robotic vacuum system under the tradename “Roomba™.”

### SUMMARY OF THE INVENTION

Accordingly, it has now been found that these and other problems found in the prior art can be overcome by an air freshener apparatus that has a source of air freshening chemical, a photocell and a fan assembly disposed in a housing adjacent the source of air freshening chemical. The fan is controlled by the optical sensor such that the fan motor is activated for a predetermined time period upon the photocell sensing a predetermined level of light. In preferred embodiments, the source of air freshening chemical is a wick, and most preferably, the wick is disposed beneath the fan and allows fragrance to be delivered over time without the fan. In certain embodiments, the air freshener also has a control circuit, or shutoff circuit that deactivates the fan motor after a predetermined time, or alternatively shuts the motor off if the sensor senses a level of light below a predetermined level, either immediately or after a predetermined length of time. The fan motor is either driven by direct current or AC line current. In the latter, in certain preferred embodiments, the housing comprises a plug that connects the motor to the AC line current via a wall outlet and a receptacle wherein the wall outlet retains its utility and can be used to power another device simultaneously with the fan.

In one aspect of certain preferred embodiments of the present invention, a microprocessor is connected to the fan motor, and drives the fan at a predetermined frequency for a predetermined duration. Most preferably, the microprocessor is connected to a micropump and to an electron spray device. In certain embodiments, the spray device is configured so that it removes particulates from the surrounding environment as part of the process of nebulizing the fragrance.

In alternate embodiments, the air freshener apparatus uses a motion sensor to control the fan. In these embodiments, the fan motor is activated for a predetermined time period upon the motion sensor being activated, and the device also has a shutoff circuit. In a manner similar to the optical sensor embodiments, the shutoff circuit either deactivates the fan motor after a predetermined time, which is either pre-set or determined by the absence of motion.

In additional embodiments, the fan-driven air freshener apparatus is integrated within a robotic vacuum cleaner assembly and is used to freshen room air as the flooring or carpets are cleaned.

### BRIEF DESCRIPTION OF THE DRAWINGS

FIG. 1 is a side elevation view of a first embodiment of a fan driven air freshener made in accordance with the present invention;

FIG. 2 is a perspective view of a second embodiment of a fan driven air freshener made in accordance with the present invention;

FIG. 3 is a perspective view of a robotic vacuum cleaner incorporating the present invention;

FIG. 4 is a perspective view of the underside of another embodiment of a robotic vacuum cleaner incorporating the present invention;

FIG. 5 is a cut away side elevation view of the robotic vacuum cleaner shown in FIG. 4;

FIG. 6 is a schematic illustrating a preferred embodiment of a control circuit for use with the present invention;

FIG. 7 is a block diagram of the operation of a fragrance dispersal system utilized in conjunction with preferred embodiments of the present invention;

FIG. 8 is partially schematic illustration of the components used in a microfluidic spray device; and

FIG. 9 is an elevation view of a micropump and capillary needle used in the device illustrated in FIG. 8.

### DETAILED DESCRIPTION OF THE PREFERRED EMBODIMENTS

The implementation of the present invention is in several preferred embodiments, discussed below, along with several illustrative examples. The embodiments of the invention described below are provided for the purpose of understanding the invention and are not meant to be limiting.

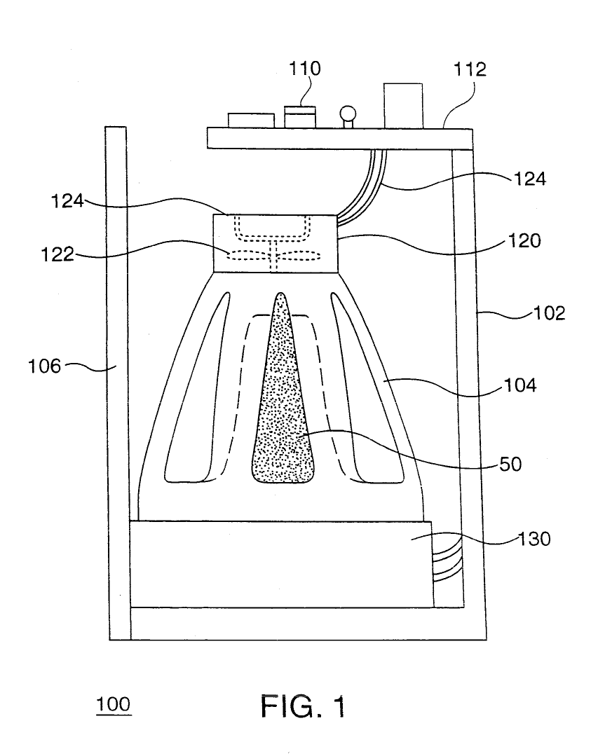

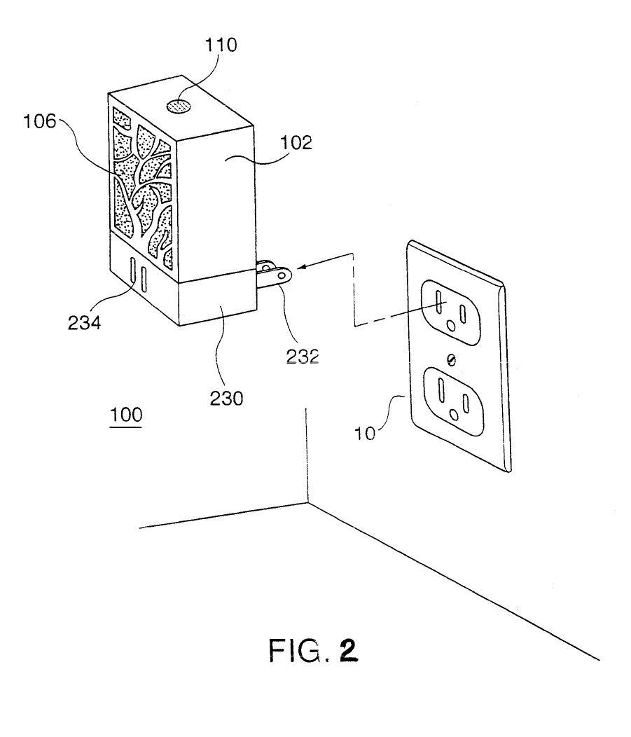

Referring now to FIG. 1, a side elevation view of a preferred embodiment of a fan driven air freshener 100 made in accordance with the present invention is illustrated. The apparatus is contained within a housing 102. In order to illustrate the invention those of skill in the art will understand that one side panel of the housing 102 has been removed. It will be further understood that the housing can be any of a number of designs and shapes, and is not limited to that which is illustrated. Generally, the housing 102 may be constructed from metal, plastic or any other suitable material that has sufficient structural strength to hold the components as shown while permitting sufficient airflow and that meets any safety or aesthetic criteria. Typically, but not necessarily, at least a portion of the housing 102 will comprise an air permeable panel 106 through which air may intermingle with air freshening chemicals. The chemicals are held in a reservoir 50, such as a wick, as is well known in the art. The chemicals may be any combination of odor masking or odor eliminating compounds that react with malodors or that have a more pleasant aroma than malodors. The composition and concentration of such chemicals for this use is well known. In a preferred embodiment, the reservoir 50 is contained within a shroud or reservoir housing 104. Preferably, the reservoir housing 104 allows the reservoir 50 to be handled without spilling or degrading the chemicals, and in certain embodiments may permit the chemicals to be replaced after they have dissipated by replacing the reservoir housing 104 and the reservoir 50 that contains fresh chemicals.
As illustrated in FIG. 1, a fan assembly 120 is preferably disposed above the reservoir 50 so as to force air through the apparatus. In certain preferred embodiments, placing the fan 120 over the reservoir 50 is preferred and is more effective than placing the fan 120 adjacent the reservoir 50. Moreover, such an embodiment can be constructed by modifying an existing air freshener assembly, which is less expensive than creating an entirely new assembly that positions the components elsewhere. The fan assembly 120 typically comprises a rotor 122 and a fan motor 124. Miniature fans suitable for any number of various embodiments of the present invention are readily available and easily adapted to the configuration shown in FIG. 1. The fan assembly 120 is driven by a power source 130. In the embodiment shown in FIG. 1 the power source 130 is preferably a direct current source, such as a battery. In addition to batteries, other conventional direct current power sources, such as solar cells, for one example, may be included in other embodiments. However, as explained in further detail below with reference to FIG. 2, the present invention also contemplates embodiments that use alternating current. The power source 130 is connected to a control circuit 112 by wires 124. As explained in further detail below, the control circuit 112 determines when the fan motor 124 is activated, and the duration of its activation.

In certain embodiments of the present invention, the control circuit 112 includes a sensor or photocell 110 that senses the level of light in the environment, and activates or deactivates a switch that supplies power to the fan motor 124. For example, the cell 110 can be chosen and put into a circuit so that the fan motor 124 is activated when a light is turned on in the room in which the apparatus is positioned. The control circuit 112 can also provide controls so that the fan 120 runs until the light is shut off, and then deactivates immediately. Alternatively, the fan 120 could run for a predetermined time (e.g., five minutes) or for a fixed time after the light source changes again, for example, when a light is turned off. The selection of a photocell and the components of the control circuit is conventional and well within the level of skill in the art. By running the fan motor 124 only when necessary, the component life is extended and the chemicals in the reservoir 50 are preserved.

Alternatively, in certain other preferred embodiments, the photocell 110 is replaced by a motion detector 110. In much the same manner as described in the preceding paragraph, the motion detector 110 determines when the fan assembly 120 should be activated, and in conjunction with the control circuit 112 determines how long a period of time the fan rotor 122 will turn. As mentioned above, the fan 120 is activated only when motion is sensed and shut off immediately in the absence of motion. Alternatively, the fan 120 can be activated when motion is sensed and then run for a fixed period. Finally, the fan can be activated and then run for a period of time measured after all motion has ceased. The selection of a motion detector and the components of the control circuit is conventional and well within the level of skill in the art.

Referring now to FIG. 2, and alternate embodiment of the present invention is illustrated. In this embodiment AC line current is used as a power source. As shown, the air freshener 100 contains an AC power supply 230 necessary to convert the line current and provide safety, if required, via a ground fault interrupter or similar circuit. The air freshener 100 has conventional plug prongs 232 that connect to a conventional receptacle 1O. In the preferred embodiment illustrated in FIG. 2, a receptacle 234 is provided that is part of the power supply assembly 230 and plug prongs 232 that connects the assembly to the power source, so that the AC power outlet retains its utility and can be used to power another device simultaneously with the fan. Alternatively, in certain embodiments, particularly those intended for non-home use, the air freshener 100 is connected directly to an AC source via a junction box or similar wiring device and is thus permanently installed in terms of the electrical connection. The alternating current embodiment illustrated in FIG. 2 is otherwise identical to that described above with reference to FIG. 1.

In accordance with on aspect of the present invention, a “burst” mode of operation is provided. It has been found that by providing a microprocessor to control the operation of the fan described above, dramatic improvement in performance can be attained. In a most preferred embodiment, the flexibility of programming a microprocessor is utilized to its fullest advantage by incorporating a micro pump into the reservoir described above and driving the pump at a first frequency, and simultaneously driving an atomizing device such as an electro sprayer at a second frequency. The selection of ideal frequencies for any particular fragrance chemical combination is routine and does not require undue experimentation. However, in any embodiment, air freshener chemical will be introduced into the air even when the fan is deactivated. Experiments have shown that adding a burst mode to the above-described device can provide 2.8 times the evaporation (i.e., a 280% increase) an effect particularly well suited for bathrooms, where it is important to modify the air for short periods of time.

| System Type | Time (hr.) | Start Wt. (g) | End Wt. (g) | Rate (g/hr.) |
|---|---|---|---|---|
| Conventional | 16.2 | 217 | 215.6 | 0.00144 |
| Fan System: | 16.2 | 58.9 | 58.4 | 0.00052 |

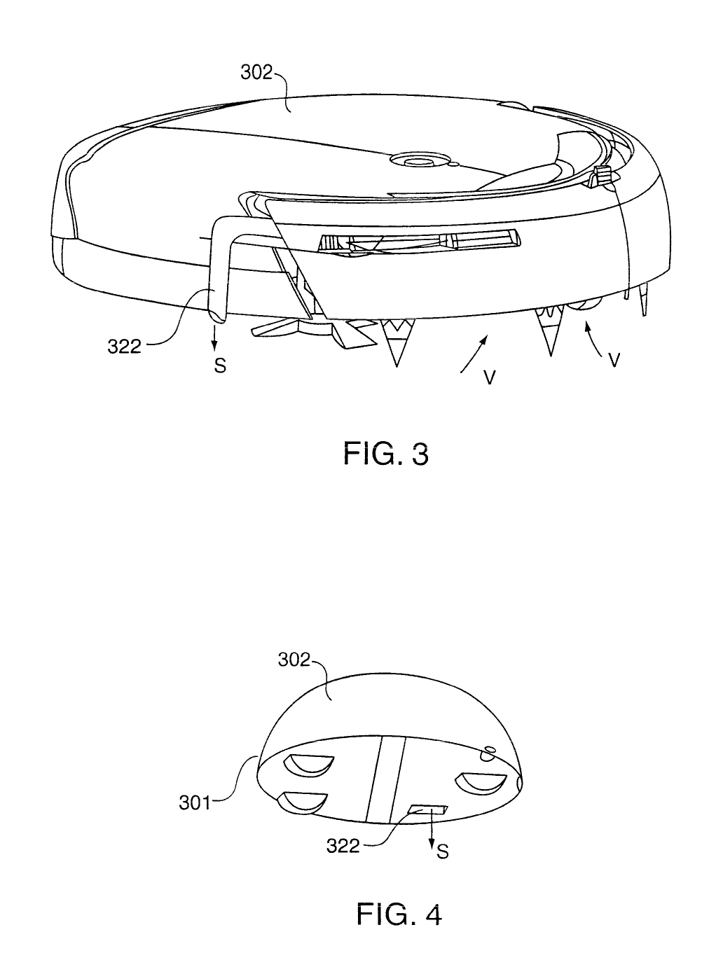

Referring now to FIGS. 3-6, additional preferred embodiments of the present invention are illustrated. In these embodiments, as shown in FIG. 3, an air freshener assembly 300 substantially as described above is integrated into a robotic vacuum cleaner 301. As readily understood by those skilled in the art, the robotic vacuum cleaner 301 is a self-contained assembly and possesses elements such as a power source, logic controls, motion sensors and fans and ducts used to create suction and lift debris from a floor or carpet surface, in the direction of the arrow marked “V” in FIG. 3. The operational parts of the robotic vacuum cleaner 301 are covered by a housing 302. In accordance with this embodiment of the present invention, an additional subsystem is added in which a fan-driven device forces a stream of scented air, in the manner shown by the arrow marked “S” in FIG. 3.

In the embodiment shown in FIG. 3, an external outlet duct 322 provides a conduit from which the scented air is forced down toward the floor. Alternatively, as illustrated in FIG. 4, the outlet duct 322 may be located internally. In either embodiment, although a single outlet duct 322 is shown, it will be understood that additional outlet ducts can be positioned in and around the structure of the robotic vacuum cleaner 301 in order to disseminate the scented air in an appropriate manner. Moreover, the outlet duct 322 may be disposed along the bottom of the unit to direct air downwardly, toward floor surfaces, such as carpeting, but in an additional embodiment is preferably oriented in other directions such as upwardly so that, for one example, a fragrance may be emitted into the room air and not necessarily on to the floor surface.

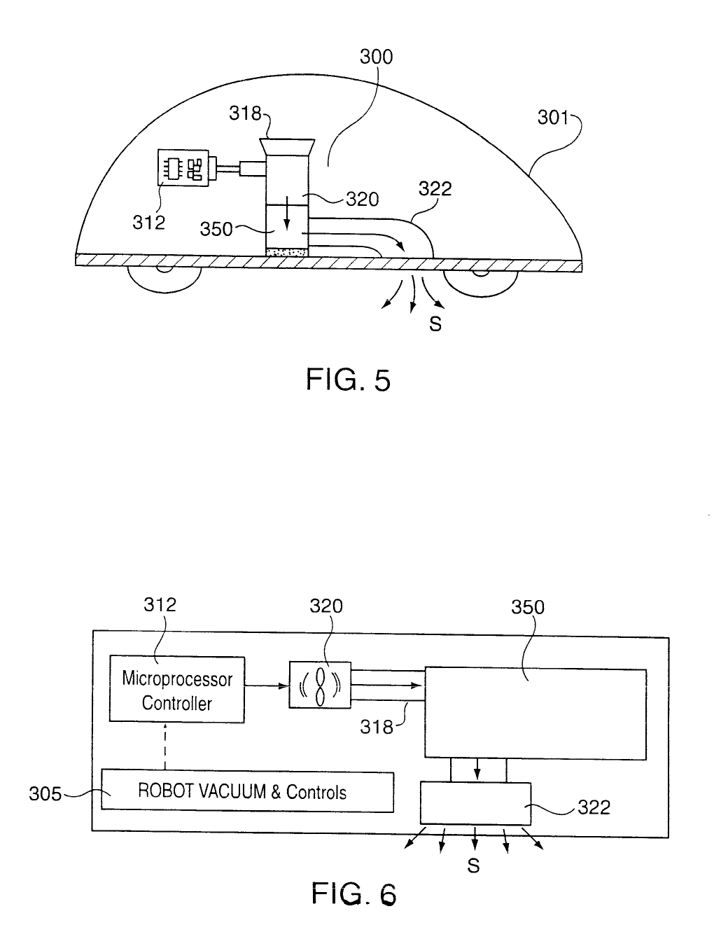

Referring now to FIG. 5, a cut away elevation view show one preferred embodiment of the present invention, in which a feed duct 318 is connected to a fan assembly 320, which forces air over and through a reservoir 350. As discussed above, the fan 320 and the reservoir 350, along with the scented material in the reservoir 350 are chosen and designed so that the rate of air flow through the reservoir 350 will volatilize an effective amount of scented chemicals so that the desired effect is created without waste or excessively rapid depletion of the reservoir 350. A stream of scented air exits the reservoir 350 via the outlet duct 322. The fan 320 may be triggered in any number of ways. As discussed above with reference to FIGS. 1-2, a triggering circuit will cause power to be delivered to a fan motor. As illustrated in FIG. 6, in certain preferred embodiments, the triggering circuit will comprise a microprocessor controller 312 that is in turn connected to and is operated by the robotic vacuum cleaner controller 305. As discussed below, the sensors and logic circuits within the robotic vacuum cleaner controller will permit the system described with reference to the present invention to be integrated into a system that enhances the operation of the robotic vacuum. The microprocessor controller in turn operates the fan 320 that volatilizes one or more chemicals in the reservoir 350 and exhausts a flow of air containing volatilized chemicals via the exhaust duct 322. Alternatively, the microprocessor controller 312 can operate a micropump and an electron spray device, as described above, to volatilize one or more chemicals in the reservoir 350, which are then exhausted through exhaust duct 322.

Thus, in the robotic vacuum cleaner embodiment illustrated in FIGS. 3-6, the signal will be provided by the microprocessor 305 that operates the other functions of the robotic vacuum cleaner unit 301. For example, the unit 301 may be programmed to first vacuum an area and then repeat its travel path while applying scented air to the cleaned surface. Alternatively, scented air could be created, either continuously or intermittently, simultaneous with the vacuuming process. In another variation the scented air applied as a separate function; e.g., the robot could vacuum floors in the morning, and then travel over the floors later in the day so that a fresh scent greets the dwelling occupant upon arrival.

Although the preferred embodiment has been described with reference to “scented air” and air freshening chemicals, those of skill in the art will appreciate that the system described herein will have applicability to a wide variety of chemical compounds that provide one or more of the following functions: cleaning, waxing, disinfecting, antibacterial, odor control, odor masking, or fragrance. In certain embodiments it will be preferable to replace the single reservoir 350 with a fragrance cartridge that contains a number of different chemicals that can be dispensed for various purposes. The cartridge can contain different scents for different rooms, or different chemicals for different purposes. For example, a cleaning chemical can be applied and then this application followed later by the application of a fragrance. In particular, using the existing programming features of robot vacuum cleaners, in certain embodiments the system disclosed herein can be “trained” to selectively deposit cleaning and/or stain inhibiting chemicals on a high traffic area, such as a doorway, since robotic vacuum cleaners have the inherent capability to “learn” the layout of a room and sense transitions between types of flooring surfaces.

In preferred embodiments, a micropump dispersal system will operate dependably in the context of the present invention without creating ozone or other undesirable atmospheric byproducts or “fallout,” yet delivering an even volumetric distribution of a fluid (i.e., a fine dispersal of fragrance) over the life of the device, or at a minimum until the volume of liquid in the reservoir is exhausted. In preferred embodiments of the present invention, a microfluidic spray device is incorporated and computer controlled to provide a fine spray of fragrance chemicals.

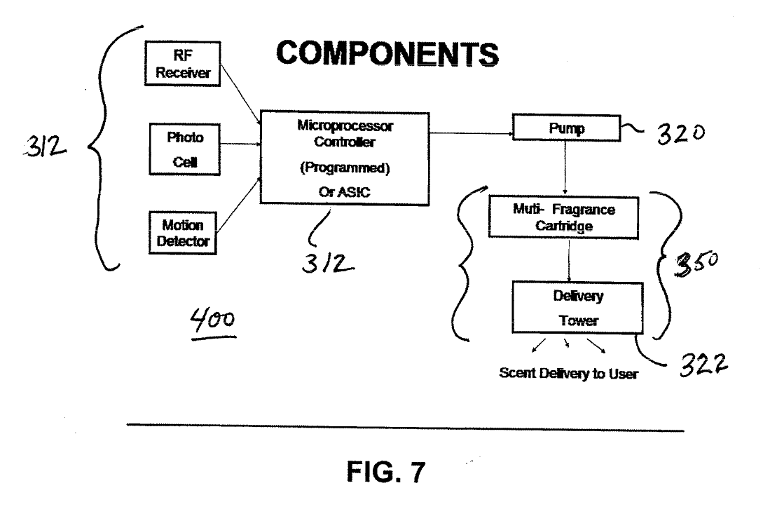

Referring to FIG. 7 a block diagram of the microfluidic dispersal system 400 integrated into certain preferred embodiments of the present invention is illustrated. An activation device 410 creates a signal that activates dispersal of one or more scents. As explained above, the activation device is typically but not necessarily an electronic signal and can be a timer or a clock, or a motion sensor or photocell. In any embodiment, a stimulus of some kind generates a signal, which in turn activates a microprocessor or an ASIC (Application Specific Integrated Circuits) 312, explained in further detail below, which operates the dispersal system. The integrated circuit in turn controls the operation of a micropump 320, which is preferably a piezoelectric device. The micropump 320 is connected to at least one source of fragrance chemical 350, but most preferably is connected to an array of fragrance chemicals, as explained below. The micropump 320 pressurizes the fragrance chemical 350 and the resulting atomized or volatilized fragrance is delivered to the environment or to the user, as explained with reference to other embodiments of the present invention. In preferred embodiments, the micropump 320 is a piezo electric device that provides constant volumetric flow over the life of the fluid supply in the fragrance chemical reservoir 350. Typically, electronic controls built into the microprocessor 312 provide precise voltage and frequency (in certain preferred embodiments the optimal frequency is 80 Hz) to the micropump 320 to deliver consistent volumetric delivery. Thus, as known in the art, the microprocessor 312 may provide for customized control of the operations of the micropump 320 and associated components, as well as providing options, such as on/off cycling, photocell operation, burst mode operation, motion activation and the like.

The microfluidic spray device is illustrated in FIG. 8. As shown, in preferred embodiments, multiple sources of fragrance chemicals (equivalent to reference numeral 350 in other figures) 440,442,444 are provided and are connected to the pump 320 in a sequence determined by the microprocessor 312. In certain embodiments, the sequence will follow directly from the activation signal. For example, if there is a scene in a film that includes a vehicle crash, the smells of engine exhaust, burning rubber, gasoline and smoke might all sequentially follow from a single activation signal in the audiovisual source. In alternate embodiments, an activation signal might initiate a longer sequence of minutes or hours where a variety of scenes are dispersed in a sequence to tell a “story” via the scents themselves. In any embodiment, it is preferred that the micropump 320 connect vial a capillary tube 434 to a charged needle dispersal valve 452, which is described in greater detail below. The charged needle system provides an output of finely divided droplets of scent chemical that disperse evenly and volatilize the scent efficiently without resort to carrier gases or high pressure. Although the system described will volatilize a liquid into a fine dispersal, it is preferable in certain embodiments to add a fan 460 to accelerate and assist in the delivery of fine fragrance to fill a room.

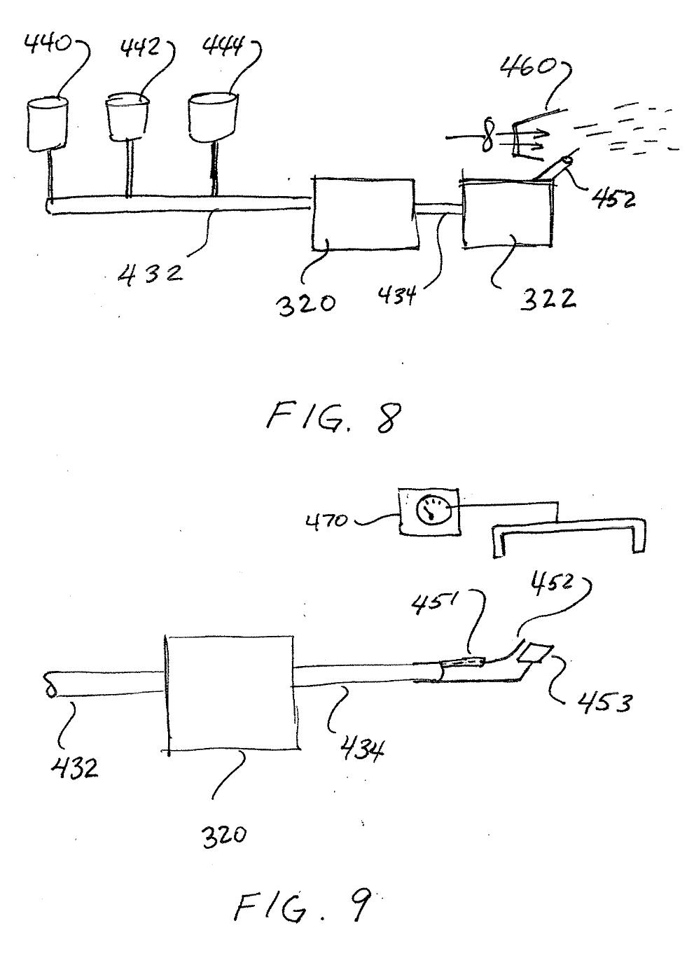

Further details of the microfluidic spray device are shown in FIG. 9. A tube 432 carries fragrance chemical into the pump 320 and, as mentioned above, a plastic capillary (tube) 434 of about 160 microns I.D. with a wall thickness of about 19 microns exits the pump 320. Inside this plastic capillary 434 is an internal (exposed to passing fluid) stainless steel metallic wire (conductor) 451 of about 19 microns O.D that terminates at its distal end as a dispersal needle 452. A positive direct current, created by 2500 volts DC or less, is applied to nebulize fine fragrance oils as well as other fluids (e.g. sanitizers such as tetraethylene glycol (TEG)) without the addition of carrier gases, which have the potential of creating ozone or other harmful byproducts. The system uses a metallic ground foil 453 to provide a target plane that attracts the positively charged nebulized fluid. In other embodiments, the ground foil is positively charged if the needle is negatively charged. This target plane 453 is preferably is maintained at a fixed distance between 0.25 inches and 0.5 inches from the exit port of the needle 452. The needle 452 is insulated from all other components such as the fragrance chemical reservoir 350 (or 440,442,444) and micropump 320. Although the length of the needle 452 is not critical to the operation, in preferred embodiments it is approximately 0.25 inches or greater in length for ease of manufacturing. To fine tune the system, it is understood by those skilled in the art that the capillary length is part of the fluidic resistance. The needle 452 is readily made longer or shorter in correspondence with the diameter of the capillary 434 to reach the desired spray volume output. Preferably, the exit port of the capillary 434 and the end of the needle 452 have a blunt end (perpendicularly cut) that optimizes the nebulization process. The internal wire (conductor), inside the capillary, ends at the blunt end of the capillary. Materials that are compatible to fine fragrance oils are used in the implementation of our prototypes. They include Dupont Kalrez®, silicone and Polyphenylsulfone (PPSU). Materials that are avoided include Polycarbonate and Polystyrene, which are poor for handling fine fragrance oils.

Although calibration of the system described herein is readily accomplished with conventional available equipment is time consuming and not very accurate, it does not require undue experimentation. However, each fragrance oil used presents another set of parameters (viscosity, conductivity, surface tension, etc) all of which affect the performance of the system and require re-calibration. Therefore, in certain preferred embodiments, the system will include a microprocessor, photodiode array, and light source disposed in the vicinity of the needle 452. The calibration system 470 is shown graphically in FIG. 9. Using discrete electronic components to make a calibration system that is dependable and accurate and provides real time measurements to permit more efficient and precise calibration. In these embodiments, the microprocessor control preferably includes a circuit design that permits the dispersal system describe herein to perform reliably and consistently. Those embodiments using ASIC's (Application Specific Integrated Circuits) will permit miniaturization of the device allowing for battery powered embodiments. The newly available Chip MAX668 along with a Microprocessor (PIC18F1220) and new software algorithms programmed into the PIC provide an optional Sine Wave or Square wave to drive the Micropump (Piezo) through Pulse Wave Modulation (PWM).

The above-described system is integrated into the vacuum cleaner apparatus described above. As shown in FIG. 6, the microprocessor 312 is controlled by and integrated with the control circuitry of the vacuum 305. As described above, these electronic controls operate the pump 320 and fragrance reservoir or cartridge 350 that is in turn connected to a delivery system 322 that preferably includes the dispersal system described above. As a result, the vacuum can now create a pattern of scents as it performs its tasks, and in certain embodiments where the fragrance is supplemented by another chemical, it can perform other tasks such as the dispersal of anti-bacterial or disinfecting fluid on surfaces, or emit certain scents based on the time of day the vacuum is operational, or emit certain scents that vary as the vacuum traverses from room to room. An advantage of the electrostatic systems described above is that allergens and the like are destroyed when in the discharge of the device. When high voltage is applied to fragrance oils, which include for example TEG (triethylene glycol) an attraction to airborne particles results causing them to precipitate. Thus one mechanism of action is that if the fragrance contains TEG or similar compounds, bacteria would become attached to these molecules and be destroyed as the oils are nebulized by electrostatic action. In addition to bacteria, other organic (or possibly even inorganic) particulates such as tobacco smoke, dander and the like can be beneficially removed from the airflow by the electrostatic nebulization process described above.

Upon review of the foregoing, numerous adaptations, modifications, and alterations will occur to the reviewer. These will all be, however, within the spirit of the present invention. Accordingly, reference should be made to the appended claims in order to ascertain the true scope of the present invention.

What is claimed is:

1. An air freshener apparatus for a robotic vacuum cleaner comprising:
a reservoir containing a chemical;
a triggering circuit controlled by the robotic vacuum cleaner;
a fragrance delivery system comprising a plurality of fragrance chemicals volatilized by a microfluidic dispersal system;
a fan assembly comprising a fan and a fan motor disposed in a housing adjacent the reservoir that is controlled by the triggering circuit; and
an outlet duct directing scented air from the fan assembly,
wherein the fragrance delivery system is by the triggering circuit.

2. The apparatus of claim 1, wherein the fragrance delivery system further comprises a capillary tube and a dispersal needle.

3. The apparatus of claim 2, wherein the dispersal needle is electrically charged.

4. The apparatus of claim 1, wherein said plurality of fragrance chemicals are connected by a the micropump via a common conduit.

5. The apparatus of claim 1, further comprising a calibration system disposed adjacent the fragrance delivery system.

6. The apparatus of claim 1, further comprising a microprocessor controller for controlling the sequence in which said fragrances are dispersed.

7. The apparatus of claim 6, wherein the microprocessor controller is triggered by an activation signal.

8. The apparatus of claim 7, wherein the microprocessor controller is an ASIC.

9. The apparatus of claim 7 wherein a sequence of fragrances is dispersed after the activation signal is received.

10. The apparatus of claim 1, wherein the chemical contained in the reservoir is an air freshening chemical.

11. The apparatus of claim 1, wherein the chemical contained in the reservoir is an anti-bacterial or anti-microbial chemical.

12. The apparatus of claim 1, wherein the chemical contained in the reservoir is an odor-masking chemical

13. The apparatus of claim 3, wherein the chemical includes one or more constituents that attract particulate matter when exposed to the dispersal needle.

14. A method of delivering into an environment a substance chosen from one or more of a fragrance or antibacterial agent comprising the steps of spraying the substance using a micropump, passing a discharge stream through an electrostatic spray device to create a delivery stream, controlling the micropump and spray device with a n electronic device.

15. The method of claim 14, wherein the electronic device is one of a microprocessor or ASIC.

16. The method of claim 14, wherein the micropump is a piezoelectric pump and further comprising the step of removing particulates from the environment.
